(19) United States
(12) Patent Application Publication
(10) Pub. No.: US 2002/0139374 A1
(43) Pub. Date: Oct. 3, 2002

(54) Safety shield

(76) Inventors: Susan A. Iliff, Scotch Plains, NJ (US); Richard Bilka, Holmdel, NJ (US); Robert C. Hill, Old Bridge, NJ (US); Gregory W. King, Carteret, NJ (US); John Kovacs, Avenel, NJ (US); Alison A. Kulick, Parlin, NJ (US); Robert K. Lang, Parlin, NJ (US); James J. Michels, Bloomfield, NJ (US); Lisa M. Stanislawczyk, Monroe Township, NJ (US)

Correspondence Address: MERCK AND CO INC, P O BOX 2000, RAHWAY, NJ, 070650907

(21) Appl. No.: 09/928,679

(22) Filed: Aug. 13, 2001

Related U.S. Application Data
(60) Provisional application No. 60/280,746, filed on Apr. 2, 2001.

(51) Int. Cl. A61F 13/00

(52) U.S. Cl. 128/846

(57) ABSTRACT

A safety shield suitable for removable attachment to a restraint chair. The shield, adjustable movably in a vertical position, characterized as a rigid, planar member adapted for removable attachment to a support member of an animal, restraining chair.

### BACKGROUND OF THE INVENTION

The safety of laboratory personnel can be compromised when working with conscious non-human primates (NHPs); particularly so when NHPs are not sedated. Injuries to laboratory personnel can occur when attempting to collect specimens from NHPs due to the animal's size and strength.

Restraint chairs and other devices have been developed to protect personnel from injuries when working with NHPs. These chairs are typically designed to include restraining panels to reduce the animal's movement. However, due to the animal's strength and defensive posture, some movement can still occur. This movement can sometime result in the injury of laboratory personnel.

It is a desire to improve the protection of laboratory personnel that works with NHPs, while not overly restraining an NHP so as to possibly injure the animal. It is also a desire to improve the performance of typical NHP restraint chairs. To that end, it is a desire to provide a device to serve as a barrier for the protection of research personnel for direct physical contact with an NHP. The ideal shield should be lightweight, easily attachable to a restraint chair, stable and quiet when in position, and able to withstand manipulation by the NHP.

### SUMMARY OF THE INVENTION

The present invention is directed to a safety shield suitable for protecting laboratory personnel from injury when working with animals, adapted for easy attachment to an animal, restraint chair. The shield being characterized as a rigid, planar member adapted for removable attachment to a support member of an animal, restraining chair, wherein the shield can be vertically adjusted and placed on different sides of the chair.

### BRIEF DESCRIPTION OF THE DRAWINGS

FIG. 1 illustrates an isometric view in elevation of the safety shield of the invention attached to a horizontal cross member of a chair; and

FIG. 2 illustrates an identical view in elevation of the safety shield with hidden views of various components of the invention.

### DETAILED DESCRIPTION OF THE INVENTION

The shield of the present invention is adapted to attach onto a macaque restrainer or a baboon restrainer, i.e. non-human primate (NHP) chair, similar to that marketed by Primate Products, Inc. of Miami, Fla. 33166 as well as other manufacturers. The shield is designed to provide protection to human, laboratory or research personnel accessing the legs of a NHP while the NHP is in the restrainer. The restrainer will generally contain vertical and horizontal cross members constructed of steel or other alloy tubing, wherein the tubing can exhibit a diameter of from about 1- to about 4-inches. The cross members on various sides of the chair are suitable for the shield to be placed in any vertical position (the plane optionally containing a handle) onto the chair. With the shield in place, NHPs will have limited access to the face, arms and hands of laboratory personnel conducting procedures such as withdrawing blood samples from the animal.

In a general embodiment of the invention, the shield may be characterized as a rigid, planar member adapted for removable attachment to a supporting cross member of an animal, restraining chair, wherein the shield is vertically or horizontally adjustable. Generally, the rigid, planar member may be constructed of a material selected from metal alloys, woods, polymeric compositions, or combinations thereof, and can have a rectangular or elliptical shape. Typically, the material of construction will be a stainless steel alloy or polyacetal suitable for sterilization; preferably, stainless steel. The supporting cross member of the chair will generally be a vertical or horizontal cross member, and the adjustable movement is performed by a notched support attached to the rigid, planar surface. The notched support member may be characterized by a plurality of vertically aligned notches suitable for removably connecting to the cross member of the chair. The notched support will contain as many notches as are suitable for vertically and horizontally moving the shield. The safety shield may further contain locking means suitable for maintaining the position of the shield on the chair support. The locking means may have a handle attached thereto for vertical and horizontal adjustments and removal of the shield from the chair.

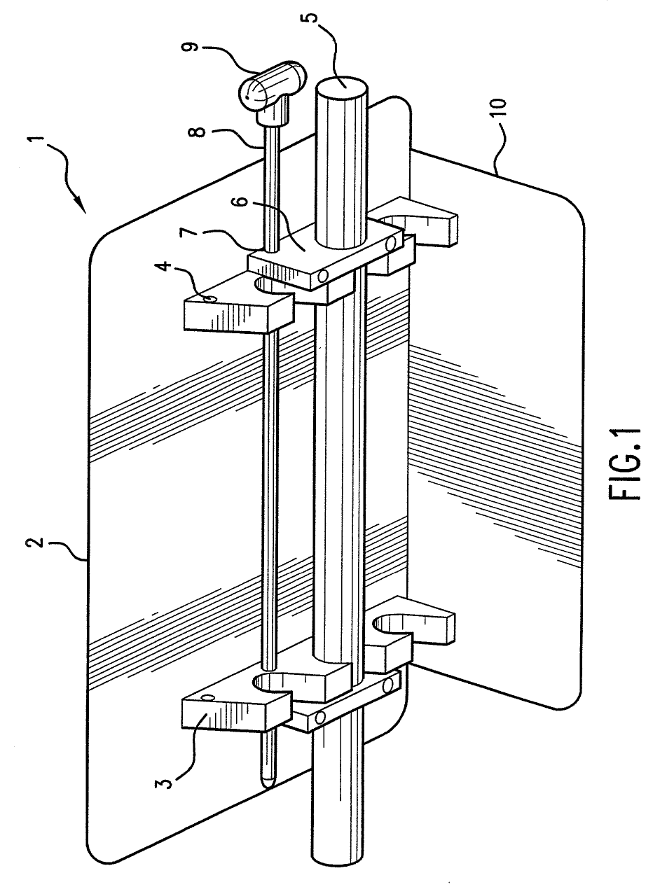

Referring to FIG. 1, an isometric view in elevation of shield 1 is shown. Rigid, planar member 2 will generally be in a vertical position when the shield is affixed onto a chair. The surface dimensions of the planar member should be sufficiently large to provide ample protection for laboratory personnel. Adjustable notched supports 3 will generally contain at least one or more vertically aligned notches for vertically positioning the shield as desired. The notched support may contain hole 4 associated with each notch for the acceptance of a locking means. The notched will be adapted to attach to supporting cross member 5 of the chair. Associated with each notched support is clamping means 6 permanently affixed the supporting cross member 5. The clamping means may contain hole 7 that aligns with hole 4 of the notched support. The notched support and clamping means will generally abut one another or be at a proximal distance, i.e. proximately close, so that when the shield is in place, the components can, together, easily accept locking means 8. Locking means 8 may be characterized as an elongated rod having handle 9 attached to one end of the rod for easy positioning through holes 4 of the notched support and 7 of the clamping means. Optionally, attached to vertical, planar member 2 may be horizontal, rigid, planar member 10. As depicted in the drawing horizontal, planar member 10 is positioned to further protect personnel from injury by the limbs of an NHP.

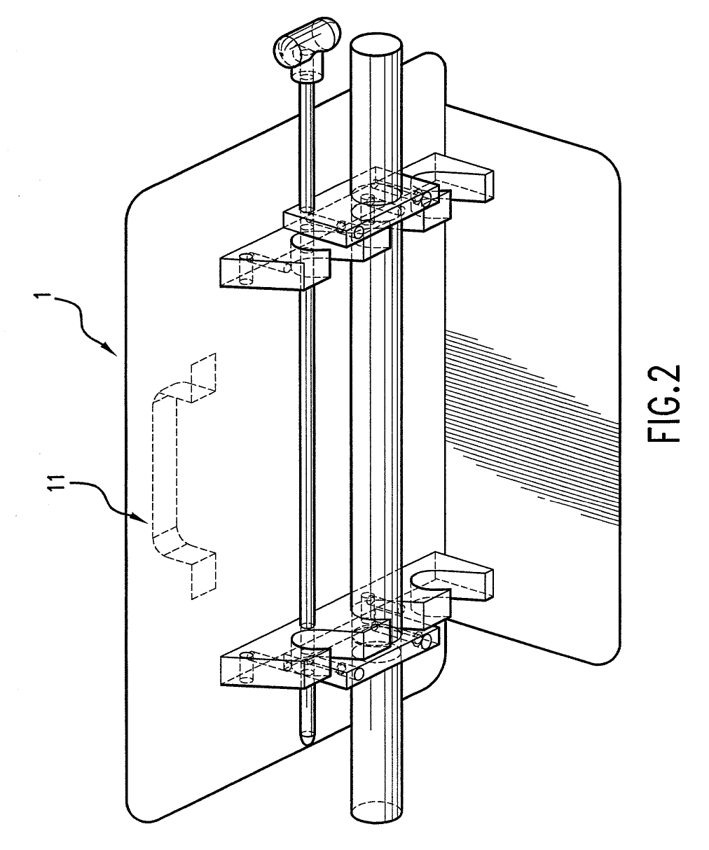

Referring to FIG. 2, there is shown an identical view as FIG. 1, however components of the shield hidden therein are illustrated as ‘dotted lines.’ Handle 11 is attached to the first surface of planar member 1 for easy handling, vertical adjustment, placement and removal of the shield from the chair. The other components of the invention as referenced in FIG. 1 may be viewed in more detail in FIG. 2.

In another embodiment of the invention, the safety shield may be, characterized as a) a vertically positioned, rigid, planar member having first and second surfaces, top and bottom sides, and left and right ends; b) at least one adjustable, notched supports attached to the second surface of the planar member for vertical adjustment thereof, wherein the notched supports adapted for receiving locking means, and wherein the supports are adapted for removable attachment to a horizontally positioned cross member of a restraint chair; c) clamping means, attached to a horizontally positioned cross member of the restraint chair and proximally abuts the adjustable support, the clamping means adapted for receiving a locking means; and d) locking means suitable for connecting the notched supports and clamping means so as to hold the planar member in a fixed position, wherein the shield is suitable for protecting laboratory personnel from injury when working with non-human primates. The notched supports, will be vertically positioned and of proximal (closely located) distance to the left and right edges of the shield, each notched support having an equal number of notches thereon, e.g. at least one notch per support; typically at least two or three, to fit onto a cross member of the chair, wherein holes through the notched support are proximal to each notch, the hole being suitable for accepting the locking means. The clamping means may be characterized as two rectangular brackets attached to the cross member of the chair, wherein each bracket has a hole therein that aligns with the holes of the notched supports positioned on the cross member of the chair, wherein the brackets are positioned at a distance of about the width of the shield, so as to closely fit next to either side of the notched support. The locking means may be characterized as an elongated rod having first and second ends, wherein a handle may be attached on the first end, and wherein the rod is suitable for removably positioning into the holes of the brackets and notched supports. Optionally, the vertical, rigid, planar member may have a horizontal, planar member attached thereto, wherein the length of the horizontal planar member extends from the left side to the right ends, and the width of the horizontal planar member is proximal to the width of the notched supports or other suitable distance to provide additional protection to a worker.

In a preferred embodiment, the safety shield, is characterized as a) a vertically positioned, rigid, planar member having first and second surfaces, top and bottom sides, and left and right ends, wherein the rigid planar member is fabricated from a material of steel alloys; b) two adjustable, notched supports attached to the second surface of the shield for vertical adjustment thereof, the notched supports adapted for receiving locking means, wherein the notched supports, are characterized as vertically attached members positioned proximal to the left and right ends of the shield, each member having an equal number of aligned notches thereon adapted to fit onto the supporting cross member of the chair, wherein the notched supports are fabricated from a polyacetal composition, and wherein a hole through the support is proximal to each notch, the hole being suitable for accepting locking means; c) two rectangular brackets attached to the cross member of the chair, wherein each bracket has a hole therein that aligns with the holes of the notched supports, wherein the brackets are positioned at a distance of about the width of the shield so as to be of close proximal distance to either side of the notched supports, wherein the brackets are fabricated from steel or other sturdy components to prevent movement of the shield on the supporting cross member of the chair, and wherein the brackets are adapted for receiving locking means; d) locking means, comprise an elongated rod having first and second ends, wherein a handle is attached on the first end suitable, the locking means suitable for connecting the notched supports and the brackets so as to hold the shield in a fixed position; and e) a horizontal, rigid, planar member attached to the vertical, rigid, planar member, wherein the length of the horizontal, planar member extends from the left side to the right side, wherein the width of the horizontal, planar member is similar to the width of the notched supports or other suitable distance to provide additional protection, and wherein the horizontal member is fabricated of stainless steel. Optionally, a handle may be attached to the first surface of the shield for easy adjustment and removal from the cross member.

What is claimed is:

1. A safety shield suitable for protecting laboratory personnel from injury when working with animals, comprising a rigid, planar member adapted for removable attachment to a supporting cross member of an animal, restraining chair, wherein the shield is adjustable.

2. The safety shield according to claim 1, wherein the rigid, planar is constructed form a material selected from the group consisting of metal alloys, woods, polymeric compositions or combinations thereof.

3. The safety shield according to claim 2, wherein the supporting cross member, comprises a vertical and horizontal cross members of the chair.

4. The safety shield according to claim 3, wherein the rigid, planar member is adapted for adjustable movement in the vertical direction.

5. The safety shield according to claim 4, wherein the adjustable movement in the vertical direction is performed by a notched support attached to the planar member.

6. The safety shield according to claim 5, wherein the notched support member comprises a plurality of aligned notches suitable for removably connecting to the supporting member of the chair.

7. The safety shield according to claim 6, further comprising locking means suitable attaching to the notched support for maintaining the position of the shield on the cross member.

8. The safety shield according to claim 7, wherein the rigid, planar member is fabricated of stainless steel.

9. The safety shield according to claim 8, wherein the animal is a non-human primate.

10. The safety shield according to claim 9, wherein a handle is attached to the shield for adjustment and removal from the chair.

11. A safety shield, comprising: a) a vertically positioned, rigid, planar member having first and second surfaces, top and bottom sides, and left and right ends; b) at least one adjustable, notched supports attached to the second surface of the shield for vertical adjustment thereof, the support adapted for receiving locking means, and wherein the support is adapted for removable attachment to a horizontally positioned supporting cross member of a restraint chair; c) clamping means, attached to a horizontally positioned supporting cross member of the chair and proximally abutting the notched support, the clamping means adapted for receiving locking means; and d) locking means suitable for connecting the notched supports and the clamping means so as to hold the shield in a fixed position, wherein the shield is suitable for protecting laboratory personnel from injury when working with non-human primates.

12. The safety shield according to claim 11, wherein the rigid, planar member is fabricated from a material selected from the group consisting of metals, wood, polymeric compositions or combinations thereof.

13. The safety shield according to claim 12, wherein the notched supports, comprise two vertically attached members positioned proximal close to the left and right ends of the shield, each member having an equal number of aligneed notches thereon adapted to fit onto the supporting cross member, wherein a hole through the support is proximally close to each notch, the hole suitable for accepting the locking means.

14. The safety shield according to claim 13, wherein the clamping means, comprise two rectangular brackets attached to the supporting cross member, wherein each bracket has a hole therein that aligns with the holes of the notched supports, and wherein the brackets are positioned at a distance of about the width of the shield.

15. The safety shield according to claim 14, wherein the locking means, comprise an elongated rod having first and second ends, wherein a handle is attached on the first end, and wherein the rod is suitable for removably positioning into the holes of the brackets and notched supports.

16. The safety shield according to claim 15, wherein the equal number of aligned notches is about three.

17. The safety shield according to claim 16, wherein the vertical, rigid, planar member has a horizontal, rigid, planar member attached thereto, wherein the length of the horizontal planar member extends from the left side to the right ends, and the width of the horizontal planar member is proximal to the width of the notched supports.

18. A safety shield, comprising: a) a vertically positioned, rigid, planar member having first and second surfaces, top and bottom sides, and left and right ends, wherein the rigid planar member is fabricated from a material selected from the group consisting of metals, wood, polymeric compositions, and combinations thereof; b) two adjustable, notched supports attached to the second surface of the shield for vertical adjustment thereof, the notched supports adapted for receiving locking means, wherein the notched supports are vertically attached to the vertically positioned, rigid, planar member positioned proximal close to the left and right ends of the shield, each notched support having an equal number of notches thereon adapted to fit onto the cross member of the chair, wherein a hole through the support is proximal close each notch, the hole being suitable for accepting a locking means; c) two rectangular brackets attached to the supporting cross member of the chair, wherein each bracket has a hole therein that aligns with the holes of the notched supports, wherein the brackets are positioned at a distance of about the width of the shield, and wherein the brackets are adapted for receiving locking means; d) locking means, comprise an elongated rod having first and second ends, wherein a handle is attached on the first end suitable for removably connecting the notched supports and the brackets so as to hold the shield in a fixed position; and e) a horizontal, rigid, planar member attached to the vertical, rigid, planar member, wherein the length of the horizontal planar member extends from the left side to the right ends of the vertical, rigid, planar member, and the width of the horizontal, planar member is proximal close to the width of the notched supports, wherein the shield is suitable for protecting laboratory personnel from injury when working with non-human primates.

19. The shield according to claim 18, wherein a handle is attached to the first side thereof.

20. The shield according to claim 19, wherein the vertical and horizontal rigid, planar members are fabricated of stainless steel.
